SEQUENCE LISTING (22 sequences)

SEQ ID NO: 1 - DNA, 1197 bp, Macaca mulatta
atggctcaaaggcagcctcactcacctaatcagactttaatttcaatcacaaatgacacagaatcaagctctgtggtttctaacgataacacaaataaaggacggagcggggacaactctccaggaatagaagcattgtgtgccatctatattacttatgctgtgatcatttcagtgggcatccttggaaatgctattctcatcaaagtctttttcaagaccaaatccatgcaaacagttccaaatattttcatcaccagcctggcttttggagatcttttacttctgctaacttgtgtgccagtggatgcaacccactaccttgcagaaggatggctgttcggaagaattggttgtaaggtgctctctttcatccggctcacttctgttggtgtgtcagtgttcacgttaacaattctcagcgctgacagatacaaggcagttgtgaagccacttgagcgacagccctccaatgccatcctgaagacttgtataaaagctggctgcgtctggatcgtgtctatgatatttgctctacctgaggctatattttcaaatgtatattcttttcgagatcccaacaaaaatgtgacatttgaatcgtgtacctcttatcctgtctctaagaagctcttgcaagaaatacattctctgctgtgcttcttagtgttctacattattccactctctattatctctgtctattattctttgattgctaggaccctttataaaagcaccctgaacatacctactgaggaacaaggccatgcccgtaagcagattgaatcccggaagagaattgccagaacggtattggtgttggtggctctgtttgccctctgctggttgccaaatcacctcctgtacctctaccattcattcacttctcaaacctatgtagacccctctgccatgcatttcattttcaccattttctctcgggttctggctttcagcaattcttgcgtaaacccctttgctctctactggctgagcaaaaccttccagaagcattttaaagctcagttgttctgttgcaaggcagagcagcctgagcctcctgttgctgacacctctcttaccaccctggctgtgatgggaagggtcccgggcactgggaacatgcagatgtctgaaattagtgtgacctcgttccctgggtgtagtgtgaagcaggcagaggatagagtctag

SEQ ID NO: 2 - protein, 398 aa, Macacca mulatta
MAQRQPHSPNQTLISITNDTESSSVVSNDNTNKGRSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLTILSADRYKAVVKPLERQPSNAILKTCIKAGCVWIVSMIFALPEAIFSNVYSFRDPNKNVTFESCTSYPVSKKLLQEIHSLLCFLVFYIIPLSIISVYYSLIARTLYKSTLNIPTEEQGHARKQIESRKRIARTVLVLVALFALCWLPNHLLYLYHSFTSQTYVDPSAMHFIFTIFSRVLAFSNSCVNPFALYWLSKTFQKHFKAQLFCCKAEQPEPPVADTSLTTLAVMGRVPGTGNMQMSEISVTSFPGCSVKQAEDRV

SEQ ID NO: 3 - DNA, 33 bp, Artificial Sequence
  PCR Primer
ttggacgtgacaatcactgtatttgaactgaga

SEQ ID NO: 4 - DNA, 27 bp, Artificial Sequence
  PCR Primer
tgtttctcctcccagcatgtgtatccg

SEQ ID NO: 5 - DNA, 19 bp, Artificial Sequence
  PCR Primer
gctctctttcatccggctc

SEQ ID NO: 6 - DNA, 20 bp, Artificial Sequence
  PCR Primer
ctgccttgtatctgtcagcg

SEQ ID NO: 7 - DNA, 18 bp, Artificial Sequence
  PCR Primer
catgcccgtaagcaggtt

SEQ ID NO: 8 - DNA, 19 bp, Artificial Sequence
  PCR Primer
cagcagagggcaaacagag

SEQ ID NO: 9 - DNA, 42 bp, Artificial Sequence
  PCR Primer
atgggatccgccaccatggctcaaaggcagcctcactcacct

SEQ ID NO: 10 - DNA, 38 bp, Artificial Sequence
  PCR Primer
atgctcgagtggaaagctagactctatcctctgcctgc

SEQ ID NO: 11 - DNA, 25 bp, Artificial Sequence
  PCR Primer
gaaagagagcaccttacaaccaatt

SEQ ID NO: 12 - DNA, 20 bp, Artificial Sequence
  PCR Primer
ccagtggatgcaacccacta

SEQ ID NO: 13 - DNA, 25 bp, Artificial Sequence
  PCR Primer
ttccgaacagccatccttctgcaag

SEQ ID NO: 14 - DNA, 23 bp, Artificial Sequence
  PCR Primer
gcaagcaggagtatgacgagtct

SEQ ID NO: 15 - DNA, 25 bp, Artificial Sequence
  PCR Primer
aactaagtcacagtccgcctagaag

SEQ ID NO: 16 - DNA, 23 bp, Artificial Sequence
  PCR Primer
cccttccatcgtccaccgcaaat

SEQ ID NO: 17 - DNA, 1200 bp, Homo Sapiens
atggctcaaaggcagcctcactcacctaatcagactttaatttcaatcacaaatgacacagaatcatcaagctctgtggtttctaacgataacacaaataaaggatggagcggggacaactctccaggaatagaagcattgtgtgccatctatattacttatgctgtgatcatttcagtgggcatccttggaaatgctattctcatcaaagtctttttcaagaccaaatccatgcaaacagttccaaatattttcatcaccagcctggcttttggagatcttttacttctgctaacttgtgtgccagtggatgcaactcactaccttgcagaaggatggctgttcggaagaattggttgtaaggtgctctctttcatccggctcacttctgttggtgtgtcagtgttcacattaacaattctcagcgctgacagatacaaggcagttgtgaagccacttgagcgacagccctccaatgccatcctgaagacttgtgtaaaagctggctgcgtctggatcgtgtctatgatatttgctctacctgaggctatattttcaaatgtatacacttttcgagatcccaataaaaatatgacatttgaatcatgtacctcttatcctgtctctaagaagctcttgcaagaaatacattctctgctgtgcttcttagtgttctacattattccactctctattatctctgtctactattccttgattgctaggaccctttacaaaagcaccctgaacatacctactgaggaacaaagccatgcccgtaagcagattgaatcccgaaagagaattgccagaacggtattggtgttggtggctctgtttgccctctgctggttgccaaatcacctcctgtacctctaccattcattcacttctcaaacctatgtagacccctctgccatgcatttcattttcaccattttctctcgggttttggctttcagcaattcttgcgtaaacccctttgctctctactggctgagcaaaagcttccagaagcattttaaagctcagttgttctgttgcaaggcggagcggcctgagcctcctgttgctgacacctctcttaccaccctggctgtgatgggaacggtcccgggcactgggagcatacagatgtctgaaattagtgtgacctcgttcactgggtgtagtgtgaagcaggcagaggacagattctag

SEQ ID NO: 18 - DNA, 1200 bp, Rattus Norvrgicus
atgtctcaaaggcagcctcagtcacctaatcagactttaatttccattacaaatgacacagaaacatcaagctctgccgtctccaacgatactacacctaaaggatggaccggagacaactctccaggaatagaagcactgtgtgccatctatatcacttatgctgtgatcatttcagtgggcatcctcggaaatgctatcctcatcaaagtctttttcaagactaaatccatgcaaacagttccaaatattttcatcaccagcctggcttttggagatctgttactcctgctgacttgtgtgccagtggatgcaacccactacctggcagagggatggctgtttggaaaggtcggttgtaaagtgctttccttcatccggctcacttctgtcggtgtatcagtgttcacgctgacaattctcagcgctgacagatacaaagcagtcgtgaagccacttgaacgacagccctccaatgccattctgaagacctgtgccaaagctggtggcatctggatcatggctatgatatttgctctgccagaggctatattctcaaatgtatacactttccaagatcctaacagaaacgtaacatttgaatcctgtaactcctaccctatctctgagaggcttttgcaggaaatacattctctgttgtgtttcttggtgttctacattatcccgctctcgattatctctgtctattattctttgattgccaggactctttacaaaagcaccttgaacataccgactgaggaacaaagccatgcccgaaagcagattgaatcccggaagagaattgccaaaacggtactggtgctggtggctctgttcgcactctgctggttgccgaatcacctcctgtatctctatcactcattcacttatgaaagctacgcagagccttctgatgtccctttcgttgtcaccattttctctcgggtgctggctttcagtaattcctgcgtgaacccctttgctctgtattggctgagcaagaccttccagaagcattttaaggctcagctctgctgcttcaaggcagagcagcctgaacctcctcttggtgacaccccccttaacaacctcactgtgatggggcgggttccagctactgggagtgcacacgtctctgaaattagcgtgaccctgtttagtggcagtactgccaagaaaggagaggacaaagtttag

SEQ ID NO: 19 - DNA, 1199 bp, Artificial Sequence
  BRS-3 consensus sequence
atggctcaaaggcagcctcactcacctaatcagactttaatttcaatcacaaatgacacagaacatcaagctctgtggtttctaacgataacacaaataaaggatggagcggggacaactctccaggaatagaagcattgtgtgccatctatattacttatgctgtgatcatttcagtgggcatccttggaaatgctattctcatcaaagtctttttcaagaccaaatccatgcaaacagttccaaatattttcatcaccagcctggcttttggagatcttttacttctgctaacttgtgtgccagtggatgcaacccactaccttgcagaaggatggctgttcggaagaattggttgtaaggtgctctctttcatccggctcacttctgttggtgtgtcagtgttcacgttaacaattctcagcgctgacagatacaaggcagttgtgaagccacttgagcgacagccctccaatgccatcctgaagacttgtgtaaaagctggctgcgtctggatcgtgtctatgatatttgctctacctgaggctatattttcaaatgtatacacttttcgagatcccaacaaaaatgtgacatttgaatcatgtacctcttatcctgtctctaagaagctcttgcaagaaatacattctctgctgtgcttcttagtgttctacattattccactctctattatctctgtctattattctttgattgctaggaccctttacaaaagcaccctgaacatacctactgaggaacaaagccatgcccgtaagcagattgaatcccggaagagaattgccagaacggtattggtgttggtggctctgtttgccctctgctggttgccaaatcacctcctgtacctctaccattcattcacttctcaaacctatgtagacccctctgccatgcatttcattttcaccattttctctcgggttctggctttcagcaattcttgcgtaaacccctttgctctctactggctgagcaaaaccttccagaagcattttaaagctcagttgttctgttgcaaggcagagcagcctgagcctcctgttgctgacacctctcttaccaccctggctgtgatgggaagggtcccgggcactgggagcatacagatgtctgaaattagtgtgacctcgttcactgggtgtagtgtgaagcaggcagaggacagagtctag

SEQ ID NO: 20 - protein, 399 aa, Homo Sapiens
MAQRQPHSPNQTLISITNDTESSSSVVSNDNTNKGWSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLTILSADRYKAVVKPLERQPSNAILKTCVKAGCVWIVSMIFALPEAIFSNVYTFRDPNKNMTFESCTSYPVSKKLLQEIHSLLCFLVFYIIPLSIISVYYSLIARTLYKSTLNIPTEEQSHARKQIESRKRIARTVLVLVALFALCWLPNHLLYLYHSFTSQTYVDPSAMHFIFTIFSRVLAFSNSCVNPFALYWLSKSFQKHFKAQLFCCKAERPEPPVADTSLTTLAVMGTVPGTGSIQMSEISVTSFTGCSVKQAEDRF

SEQ ID NO: 21 - protein, 399 aa, Rattus Norvegicus
MSQRQPQSPNQTLISITNDTETSSSAVSNDTTPKGWTGDNSPGIEALCAIYITYAVIISVGILGNAILIKVFFKTKSMQTVPNIFITSLAFGDLLLLLTCVPVDATHYLAEGWLFGKVGCKVLSFIRLTSVGVSVFTLTILSADRYKAVVKPLERQPSNAILKTCAKAGGIWIMAMIFALPEAIFSNVYTFQDPNRNVTFESCNSYPISERLLQEIHSLLCFLVFYIIPLSIISVYYSLIARTLYKSTLNIPTEEQSHARKQIESRKRIAKTVLVLVALFALCWLPNHLLYLYHSFTYESYAEPSDVPFVVTIFSRVLAFSNSCVNPFALYWLSKTFQKHFKAQLCCFKAEQPEPPLGDTPLNNLTVMGRVPATGSAHVSEISVTLFSGSTAKKGEDKV

SEQ ID NO: 22 - protein, 399 aa, Artificial Sequence
  BRS-3 consensus sequence
MAQRQPHSPNQTLISITNDTESSSSVVSNDNTNKGWSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLTILSADRYKAVVKPLERQPSNAILKTCIKAGCVWIVSMIFALPEAIFSNVYTFRDPNKNVTFESCTSYPVSKKLLQEIHSLLCFLVFYIIPLSIISVYYSLIARTLYKSTLNIPTEEQSHARKQIESRKRIARTVLVLVALFALCWLPNHLLYLYHSFTSQTYVDPSAMHFIFTIFSRVLAFSNSCVNPFALYWLSKTFQKHFKAQLFCCKAEQPEPPVADTSLTTLAVMGRVPGTGSIQMSEISVTSFSGCSVKQAEDRV

(19) United States
(12) Patent Application Publication
(10) Pub. No.: US 2007/0037219 A1
(43) Pub. Date: Feb. 15, 2007

(54) Rhesus monkey bombesin receptor subtype-3 (brs-3), nucleotides encoding same, and uses thereof

(76) Inventors: Carina P. Tan, Metuchen, NJ (US); Andrew D. Howard, Park Ridge, NJ (US); Hideki Sano, Tsukuba (JP)

Correspondence Address: MERCK AND CO., INC, P O BOX 2000, RAHWAY, NJ, 07065-0907, US

(21) Appl. No.: 10/553,812

(22) PCT Filed: Apr. 14, 2004
(86) PCT No.: PCT/US04/11473

§ 371 Date: Oct. 2, 2006

Related U.S. Application Data
(60) Provisional application No. 60/463,776, filed on Apr. 18, 2003.

(51) Int. Cl. G01N 33/567; C07H 21/04; C12P 21/06; C12N 5/06; C07K 14/705

(52) U.S. Cl. 435/7.2; 435/69.1; 435/320.1; 435/364; 530/350; 536/23.5

(57) ABSTRACT

A rhesus monkey bombesin receptor subtype-3 has been isolated, cloned and sequenced. This receptor is characteristic of the G-protein family of receptors. Isolated rhesus monkey bombesin receptor subtype-3 may be used to screen and identify novel bombesin receptor modulators that may contribute to the regulation of endocrine processes, metabolism, or the cell cycle. Such compounds may be used in the treatment of conditions that result from deregulated expression of bombesin receptor subtype-3.

### CROSS-REFERENCE TO RELATED APPLICATIONS

This application claims the benefit of U.S. Provisional Application No. 60/463,776, filed Apr. 18, 2003, the contents of which are incorporated herein by reference in their entirety.

### FIELD OF THE INVENTION

The present invention relates to rhesus monkey bombesin receptor subtype-3, herein designated rhBRS-3, to isolated nucleic acid molecules which encode this receptor, to recombinant vectors and hosts comprising DNA encoding this receptor and to use of rhBRS-3 in various assays.

### BACKGROUND OF THE INVENTION

Bombesin, a tetradecapeptide originally isolated from frog skin, represents the first member of a large family of regulatory peptides named bombesin-like peptides. Two bombesin-like peptides, gastrin-releasing peptide (GRP) (McDonald et al., Biochem. Biophys. Res. Commun. 90: 227-33 (1979)) and neuromedin B (NMB) (Minamino et al., Biochemn. Biophys. Res. Commun. 114: 541-548 (1983)) have been found in mammals.

Bombesin receptor subtype-3 (BRS-3, also named BB3) is one of three subtypes of bombesin receptors, which was identified based on its high degree of homology to mammalian bombesin receptors. BRS-3 is a member of the G protein-coupled receptor superfamily and has been cloned from human, mouse and sheep (Whitley et al., J. Mol. Endocrinol. 23: 107-16 (1999)). A naturally occurring high affinity ligand for BRS-3 has not been identified. However, a synthetic peptide, [D-Tyr6-betaAlal 11-Phe13-Nle14] bombesin(6-14) (hereinafter dYB) was shown to have high affinity for all three human bombesin receptor subtypes (Pradhan et al., Eur. J. Pharmacol. 343: 275-87 (1998)).

The human BRS-3 sequence was originally described by Fathi et al. (J. Biol. Chem. 268(8): 5979-84 (1993)). A variant of the human sequence was also described (U.S. Pat. No. 6,143,521).

In addition to the human clones described above, rat BRS-3 sequences were disclosed by Liu et al (WO 03/014310) and by Spindel et al. (U.S. Pat. No. 5,656,749). A truncated BRS-3 was also isolated from rat by Lane et al. (WO 01/10889).

Bombesin, bombesin-like peptides and related receptors participate in a diverse array of physiological processes. BRS-3 has been implicated in the regulation of neuroendocrine function and energy metabolism (Ohki et al. Nature 390: 165-69 (1997)). Mice lacking functional BRS-3 are hyperphagic and have a reduced metabolic rate, which leads to the development of obesity, hypertension and diabetes as adults. Additionally, bombesin-like peptides may contribute to the pathogenesis of some human carcinomas (For review, see Lebacq-Verheyden et al., in Handbook of Experimental Pharmacology, Sporn, M. N. and Roberts, A. B., eds., Vol. 95, pp. 71-124, Springer-Verlag, Berlin).

Despite the identification of the cDNA clones encoding bombesin receptor subtypes mentioned above, it would be advantageous to identify additional mammalian genes encoding bombesin receptor subtypes in order to allow screening to identify novel bombesin receptor modulators that may contribute to the regulation of endocrine processes, metabolism, or the cell cycle.

### SUMMARY OF THE INVENTION

The present invention relates to an isolated or purified nucleic acid molecule (polynucleotide) which encodes a novel rhesus monkey bombesin receptor subtype-3 (hereinafter rhBRS-3). The DNA molecules disclosed herein may be transfected into a host cell of choice wherein the recombinant host cell provides a source for substantial levels of an expressed functional rhBRS-3 protein (SEQ ID NO:2). This receptor protein provides a screening target to identify modulators of bombesin and bombesin-like peptides, which may be involved in the pathogenesis of a variety of human disorders when deregulated.

The present invention also relates to isolated nucleic acid molecules comprising a sequence of nucleotides that encode a rhesus monkey BRS-3 protein as set forth in SEQ ID NO:2. In an exemplary embodiment of this aspect of the invention, the nucleic acid molecule comprises a sequence of nucleotides as set forth in SEQ ID NO:1.

Included in this invention are biologically active fragments or mutants of SEQ ID NO:1, which encode mRNA expressing a novel rhBRS-3 protein. Any such biologically active fragment and/or mutant will encode either a protein or protein fragment which at least substantially mimics the pharmacological properties of the rhBRS-3 protein, including but not limited to the rhBRS-3 protein as set forth in SEQ ID NO:2.

The present invention further relates to a process for expressing a rhesus monkey BRS-3 protein in a recombinant host cell, comprising: (a) introducing a vector comprising an isolated nucleic acid molecule into a suitable host cell, the nucleic acid molecule comprising a sequence of nucleotides that encodes a rhesus monkey BRS-3 protein as set forth in SEQ ID NO:2; and, (b) culturing the host cell under conditions which allow expression of said rhesus monkey BRS-3 protein.

The present invention also relates to recombinant vectors and recombinant host cells, both prokaryotic and eukaryotic, which contain the nucleic acid molecules disclosed throughout this specification.

Another aspect of the present invention is a substantially purified form of a rhesus monkey BRS-3 protein which consists of the amino acid sequence disclosed in FIG. 2 (SEQ ID NO:2). Characterization of the BRS-3 protein will allow for screening to identify novel bombesin receptor subtype-3 modulators that may have a role in the regulation of endocrine processes or metabolism. As noted above, heterologous expression of rhesus monkey BRS-3 disclosed herein is contemplated at levels substantially above endogenous levels and will allow for the pharmacological analysis of compounds which may contribute to the pathogenesis of a variety of human disorders associated with deregulated BRS-3 expression. Heterologous cell lines expressing a functional rhesus monkey BRS-3 (e.g., functional forms of SEQ ID NO: 2), can be used to establish functional or binding assays to identify novel BRS-3 modulators that may be useful in the development of therapeutics for human diseases associated with deregulated BRS-3 expression.

The present invention also provides biologically active fragments and/or mutants of a rhesus monkey BRS-3 protein, comprising the amino acid sequence as set forth in SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for selective modulators, including but not limited to agonists and/or antagonists for rhesus monkey bombesin and bombesin-like peptide receptor pharmacology.

The present invention also relates to a substantially purified, fully processed (including proteolytic processing, glycosylation and/or phosphorylation), mature BRS-3 protein obtained from a recombinant host cell containing a DNA expression vector comprising nucleotide sequence as set forth in SEQ ID NO:1, which expresses the rhBRS-3 protein.

The present invention also relates to rhesus monkey BRS-3 fusion constructs, including but not limited to fusion constructs which express a portion of the rhesus monkey BRS-3 protein linked to various markers, including but in no way limited to GFP (Green fluorescent protein), the MYC epitope, GST, and Fc. Any such fusion constructs may be expressed in the cell line of interest and used to screen for modulators of the rhesus monkey BRS-3 protein disclosed herein.

The present invention further relates to methods of expressing rhesus monkey BRS-3 proteins and biological equivalents disclosed herein, assays employing these gene products, recombinant host cells which comprise DNA constructs which express these proteins, and compounds identified through these assays which act as agonists or antagonists of BRS-3 activity.

The present invention further relates to methods for screening for compounds which modulate the expression of DNA or RNA encoding a rhBRS-3 protein as well as compounds which effect the function of the rhBRS-3 protein.

Also provided herein is a method for identifying compounds that modulate rhesus monkey bombesin receptor subtype-3 expression, comprising contacting a test compound with rhesus monkey bombesin receptor subtype-3, and determining whether the test compound interacts with rhesus monkey bombesin receptor subtype-3.

This invention further relates to a method for determining whether a substance is capable of binding to rhesus monkey BRS-3 (rhBRS-3) comprising: (a) providing test cells by transfecting cells with an expression vector that directs the expression of rhBRS-3 in the cells; (b) exposing the test cells to the substance; (c) measuring the amount of binding of the substance to rhBRS-3; and, (d) comparing the amount of binding of the substance to rhBRS-3 in the test cells with the amount of binding of the substance to control cells that have not been transfected with rhBRS-3.

Also provided herein is a method for determining whether a substance is a potential agonist or antagonist of rhBRS-3 comprising: (a) transfecting or transforming cells with an expression vector that directs expression of rhBRS-3 in the cells, resulting in test cells; (b) allowing the test cells to grow for a time sufficient to allow rhBRS-3 to be expressed; (c) exposing the cells to a labeled ligand of rhBRS-3 in the presence and in the absence of the substance; and, (d) measuring the binding of the labeled ligand to rhBRS-3; where if the amount of binding of the labeled ligand is less in the presence of the substance than in the absence of the substance, then the substance is a potential agonist or antagonist of rhBRS-3.

Another preferred aspect of the present invention is a substantially purified membrane preparation, partially purified membrane preparation, or cell lysate which has been obtained from a recombinant host cell transformed or transfected with a DNA expression vector which comprises and appropriately expresses a complete open reading frame as set forth in SEQ ID NO:1, resulting in a functional form of rhBRS-3.

As used throughout the specification and in the appended claims, the singular forms “a,” “an,” and “the” include the plural reference unless the context clearly dictates otherwise.

As used throughout the specification and appended claims, the following definitions and abbreviations apply:

“Substantially free from other nucleic acids” means at least 90%, preferably 95%, more preferably 99%, and even more preferably 99.9%, free of other nucleic acids. As used interchangeably, the terms “substantially free from other nucleic acids,” “substantially purified,” “isolated nucleic acid” or “purified nucleic acid” also refer to DNA molecules which comprise a coding region for a rhesus monkey BRS-3 protein that has been purified away from other cellular components. Thus, a rhesus monkey BRS-3 DNA preparation that is substantially free from other nucleic acids will contain, as a percent of its total nucleic acid, no more than 10%, preferably no more than 5%, more preferably no more than 1%, and even more preferably no more than 0.1%, of non-rhesus BRS-3 nucleic acids. Whether a given rhesus monkey BRS-3 DNA preparation is substantially free from other nucleic acids can be determined by such conventional techniques of assessing nucleic acid purity as, e.g., agarose gel electrophoresis combined with appropriate staining methods, e.g., ethidium bromide staining, or by sequencing.

“Substantially free from other proteins” or “substantially purified” means at least 90%, preferably 95%, more preferably 99%, and even more preferably 99.9%, free of other proteins. Thus, a rhesus monkey BRS-3 protein preparation that is substantially free from other proteins will contain, as a percent of its total protein, no more than 10%, preferably no more than 5%, more preferably no more than 1%, and even more preferably no more than 0.1%, of non-rhesus monkey BRS-3 proteins. Whether a given rhesus monkey BRS-3 protein preparation is substantially free from other proteins can be determined by such conventional techniques of assessing protein purity as, e.g., sodium dodecyl sulfate polyacrylamide gel electrophoresis (SDS-PAGE) combined with appropriate detection methods, e.g., silver staining or immunoblotting. As used interchangeably, the terms “substantially free from other proteins” or “substantially purified”, or “isolated rhesus monkey BRS-3 protein” or “purified rhesus monkey BRS-3 protein” also refer to rhesus monkey BRS-3 protein that has been isolated from a natural source.

Use of the term “isolated” or “purified” indicates that rhesus monkey BRS-3 protein has been removed from its normal cellular environment. Thus, an isolated rhesus monkey BRS-3 protein may be in a cell-free solution or placed in a different cellular environment from that in which it occurs naturally. The term isolated does not imply that an isolated rhesus monkey BRS-3 protein is the only protein present, but instead means that an isolated rhesus monkey BRS-3 protein is substantially free of other proteins and non-amino acid material (e.g., nucleic acids, lipids, carbohydrates) naturally associated with the rhesus BRS-3 protein in vivo. Thus, a rhesus monkey BRS-3 protein that is recombinantly expressed in a prokaryotic or eukaryotic cell and substantially purified from this host cell which does not naturally (i.e., without intervention) express this BRS-3 protein is of course “isolated rhesus monkey BRS-3 protein” under any circumstances referred to herein.

As noted above, a rhesus BRS-3 protein preparation that is an isolated or purified rhesus monkey BRS-3 protein will be substantially free from other proteins and will contain, as a percent of its total protein, no more than 10%, preferably no more than 5%, more preferably no more than 1%, and even more preferably no more than 0.1%, of non-rhesus monkey BRS-3 proteins.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

The term “rhBRS” refers to a—rhesus monkey bombesin receptor subtype-3—.

The term “mammalian” will refer to any mammal, including a human being.

### BRIEF DESCRIPTION OF THE DRAWINGS

FIG. 1 shows the nucleotide sequence of rhesus monkey BRS-3 cDNA, as set forth in SEQ ID NO:1.

FIG. 2 shows the predicted amino acid sequence of rhesus monkey BRS-3 protein, as set forth in SEQ ID NO:2.

FIG. 3 shows an alignment of the human (SEQ ID NO:17, see Fathi et al., supra), rat (SEQ ID NO:18, see Liu et al., WO 03/014310) and rhesus monkey (SEQ ID NO:1) BRS-3 nucleotide sequences. Nucleotides that are different among the BRS-3 sequences are shown in bold. Dashes indicate that spaces were added to facilitate the alignment. A consensus sequence (SEQ ID NO:19), derived by comparing the above nucleotide sequences, is also shown.

FIG. 4 shows an alignment of the human (SEQ ID NO:20, see Fathi et al., supra), rat (SEQ ID NO:21, see Liu et al., WO 03/014310) and rhesus monkey (SEQ ID NO:2) BRS-3 open reading frames. Amino acids that are different among the BRS-3 sequences are shown in bold. Dashes indicate that spaces were added to facilitate the alignment. A consensus sequence (SEQ ID NO:22), derived by comparing the above protein sequences, is also shown.

### DETAILED DESCRIPTION OF THE INVENTION

Bombesin, bombesin-like peptides and related receptors participate in a diverse array of physiological processes, including the regulation of neuroendocrine function and energy metabolism. Deregulation of normal expression patterns of bombesin receptors, including BRS-3, can lead to various human disorders such as obesity, hypertension and diabetes. Additionally, studies suggest that bombesin-like peptides can contribute to the pathogenesis of some human carcinomas. Therefore, the isolated nucleic acid molecules, associated vectors, host cells, recombinant subcellular fractions and membranes, and the expressed and mature forms of the rhesus monkey BRS-3 protein provided by the present invention are important tools for drug discovery. These embodiments of the present invention may be employed in methods for screening for compounds which modulate the expression of DNA or RNA encoding a rhBRS-3 protein as well as compounds which effect the function of the rhBRS-3 protein. Said compounds will find use in pharmaceutical compositions for the treatment and/or prevention of human disorders.

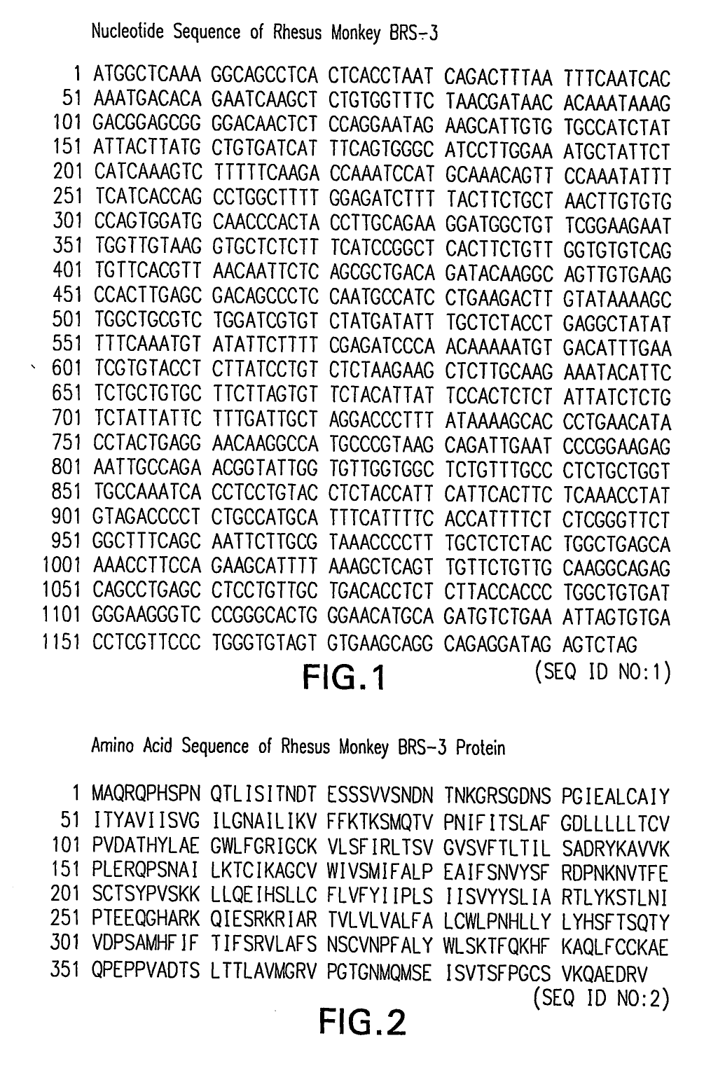

The present invention relates to an isolated nucleic acid molecule (polynucleotide) which encodes rhesus monkey bombesin receptor subtype-3 protein (SEQ ID NO:2). A preferred aspect of this portion of the present invention is disclosed in FIG. 1 (SEQ ID NO:1), which shows a DNA molecule encoding a novel rhBRS-3 protein (SEQ ID NO:2).

The isolated nucleic acid molecules of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA). For most cloning purposes, DNA is a preferred nucleic acid. The nucleic acid molecules of the present invention are substantially free from other nucleic acids.

As noted above, an exemplary embodiment of the present invention is an isolated nucleic acid molecule (polynucleotide) which encodes MRNA which expresses a novel rhesus monkey bombesin receptor subtype-3 protein, this DNA molecule comprising the nucleotide sequence disclosed herein as SEQ ID NO:1. This rhBRS-3 nucleic acid molecule was identified through RT-PCR as described in detail in EXAMPLE 1.

Included in this invention are biologically active fragments or mutants of SEQ ID NO:1, which encode mRNA expressing a novel rhBRS-3 protein. Any such biologically active fragment and/or mutant will encode either a protein or protein fragment which at least substantially mimics the pharmacological properties of the rhBRS-3 protein, including but not limited to the rhBRS-3 protein as set forth in SEQ ID NO:2. Any such polynucleotide includes but is not necessarily limited to nucleotide substitutions, deletions, additions, arnino-terminal truncations and carboxy-terminal truncations such that these mutations encode mRNA which express a functional rhBRS-3 protein in a eukaryotic cell, such as Xenopus oocytes, so as to be useful for screening for agonists and/or antagonists of rhesus monkey BRS-3 activity.

In preferred embodiments of the invention, DNA is ligated into a vector, and introduced into suitable host cells to produce transformed cell lines that express the rhesus monkey BRS-3 protein, or a fragment thereof. The resulting cell lines can then be produced in quantity for reproducible quantitative analysis of the effects of drugs on receptor function.

In other embodiments, mRNA may be produced by in vitro transcription of DNA encoding the invention peptide. This MRNA can then be injected into Xenopus oocytes where the RNA directs the synthesis of the rhesus monkey BRS-3 protein. Alternatively, the invention-encoding DNA can be directly injected into oocytes for expression of a functional invention peptide. The transfected mammalian cells or injected oocytes may then be used in the methods of drug screening provided herein.

Therefore, the heterologous expression of the rhesus monkey BRS-3 protein will allow the pharmacological analysis of compounds that may contribute to the regulation of the endocrine system, cell cycle or metabolism. Heterologous cell lines expressing these rhBRS-3 proteins can be used to establish functional or binding assays to identify novel rhBRS-3 modulators that may be useful in the development of novel human therapeutics for diseases related to deregulated bombesin receptor expression.

Another aspect of the present invention is a substantially purified form of a rhesus monkey BRS-3 protein which consists of the amino acid sequence disclosed in FIG. 2 (SEQ ID NO:2). This receptor protein provides a screening target to identify modulators of bombesin and bombesin-like peptides, which may be involved in the pathogenesis of a variety of human disorders when deregulated.

The present invention also provides biologically active fragments and/or mutants of a rhesus monkey BRS-3 protein, comprising the amino acid sequence as set forth in SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for selective modulators, including but not limited to agonists and/or antagonists for rhesus monkey bombesin and bombesin-like peptide receptor pharmacology.

The present invention also relates to a substantially purified, fully processed (including proteolytic processing, glycosylation and/or phosphorylation), mature BRS-3 protein obtained from a recombinant host cell containing a DNA expression vector comprising nucleotide sequence as set forth in SEQ ID NO:1, which expresses the rhBRS-3 protein. It is especially preferred that the recombinant host cell be a eukaryotic host cell, such as a mammalian cell line, or Xenopus oocytes, as noted above.

As noted above, a preferred aspect of the present invention is disclosed in FIG. 2 (SEQ ID NO:2), which indicates the amino acid sequence of the rhesus monkey BRS-3 protein of the present invention. Characterization of this protein will allow for screening to identify novel bombesin receptor subtype-3 modulators that may have a role in the regulation of endocrine processes or metabolism. Heterologous expression of rhesus monkey BRS-3 disclosed herein is contemplated at levels substantially above endogenous levels and will allow for the pharmacological analysis of compounds which may contribute to the pathogenesis of a variety of human disorders associated with deregulated BRS-3 expression. Heterologous cell lines expressing a functional rhesus monkey BRS-3 (e.g., functional forms of SEQ ID NO: 2), can be used to establish functional or binding assays to identify novel BRS-3 modulators that may be useful in the development of therapeutics for human diseases associated with deregulated BRS-3 expression.

The rhesus monkey BRS-3 receptor proteins of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Accordingly, the present invention relates to rhesus monkey BRS-3 fusion constructs, including but not limited to fusion constructs which express a portion of the rhesus monkey BRS-3 protein linked to various markers, including but in no way limited to GFP (Green fluorescent protein), the MYC epitope, GST, and Fc. Any such fusion constructs may be expressed in the cell line of interest and used to screen for modulators of the rhesus monkey BRS-3 protein disclosed herein.

This invention also relates to various functional domains of the rhBRS-3 receptor, such as the extracellular domain and the intracellular domain, and to hybrid molecules comprising at least one of these sequences. Accordingly, the present invention includes chimeric polypeptides wherein at least one domain of the rhesus monkey BRS-3 polypeptide is linked a non-rhesus monkey BRS-3 sequence of amino acid residues to produce a chimeric polypeptide. The present invention also includes isolated nucleic acid molecules, comprising a sequence of nucleotides that encodes said chimeric polypeptide.

As noted above, a preferred aspect of this invention is a DNA molecule described in FIG. 1 as rhesus monkey BRS-3 and set forth as SEQ ID NO:1, which encodes the rhesus monkey bombesin receptor subtype-3 protein shown in FIG. 2 and set forth as SEQ ID NO:2.

It is well understood in the art that differing DNA molecules may express an identical protein due to codon redundancy. Accordingly, this invention also relates to synthetic DNA that encodes the rhBRS-3 protein where the nucleotide sequence of the synthetic DNA differs significantly from the nucleotide sequence of SEQ ID NO:1 but still encodes the same rhBRS-3 protein as SEQ ID NO:2. Such synthetic DNAs are intended to be within the scope of the present invention. If it is desired to express such synthetic DNAs in a particular host cell or organism, the codon usage of such synthetic DNAs can be adjusted to reflect the codon usage of that particular host, thus leading to higher levels of expression of the BRS-3 protein in the host.

Therefore, the present invention discloses codon redundancy that may result in differing DNA molecules expressing an identical protein. For purposes of this specification, a sequence bearing one or more replaced codons will be defined as a degenerate variation. Also included within the scope of this invention are mutations either in the DNA sequence or the translated protein that do not substantially alter the ultimate physical properties of the expressed protein. For example, substitution of valine for leucine, arginine for lysine, or asparagine for glutamine may not cause a change in the functionality of the polypeptide.

It is known that DNA sequences coding for a peptide may be altered so as to code for a peptide that has properties that are different than those of the naturally occurring peptide. Methods of altering the DNA sequences include but are not limited to site directed mutagenesis. Examples of altered properties include but are not limited to changes in the affinity of an enzyme for a substrate or receptor for a ligand.

Any of a variety of procedures may be used to clone rhBRS-3. These methods include, but are not limited to, (1) a RACE PCR cloning technique (Frohman, et al., 1988, Proc. Natl. Acad. Sci. USA 85: 8998-9002). 5′ and/or 3′ RACE may be performed to generate a full-length cDNA sequence. This strategy involves using gene-specific oligonucleotide primers for PCR amplification of rhBRS-3 cDNA. These gene-specific primers are designed through identification of an expressed sequence tag (EST) nucleotide sequence which has been identified by searching any number of publicly available nucleic acid and protein databases; (2) direct functional expression of the rhBRS-3 cDNA following the construction of a rhBRS-3-containing cDNA library in an appropriate expression vector system; (3) screening an rhBRS-3-containing cDNA library constructed in a bacteriophage or plasmid shuttle vector with a labeled degenerate oligonucleotide probe designed from the amino acid sequence of the rhBRS-3 protein; (4) screening an rhBRS-3-containing cDNA library constructed in a bacteriophage or plasmid shuttle vector with a partial cDNA encoding the rhBRS-3 protein. This partial cDNA is obtained by the specific PCR amplification of rhBRS-3 DNA fragments through the design of degenerate oligonucleotide primers from the amino acid sequence known for other kinases which are related to the rhBRS-3 protein; (5) screening a rhBRS-3-containing cDNA library constructed in a bacteriophage or plasmid shuttle vector with a partial cDNA or oligonucleotide with homology to a mammalian rhBRS-3 protein. This strategy may also involve using gene-specific oligonucleotide primers for PCR amplification of rhBRS-3 cDNA identified as an EST as described above; or (6) designing 5′ and 3′ gene specific oligonucleotides using SEQ ID NO:1 as a template so that either the full-length cDNA may be generated by known RACE techniques, or a portion of the coding region may be generated by these same known RACE techniques to generate and isolate a portion of the coding region to use as a probe to screen one of numerous types of cDNA and/or genomic libraries in order to isolate a full-length version of the nucleotide sequence encoding rhBRS-3.

It is readily apparent to those skilled in the art that other types of libraries, as well as libraries constructed from other cell types-or species types, may be useful for isolating a rhBRS-3-encoding DNA or a rhBRS-3 homologue. Other types of libraries include, but are not limited to, cDNA libraries derived from other cells.

It is readily apparent to those skilled in the art that suitable cDNA libraries may be prepared from cells or cell lines which have rhBRS-3 activity. The selection of cells or cell lines for use in preparing a cDNA library to isolate a cDNA encoding rhBRS-3 may be done by first measuring cell-associated rhBRS-3 activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

The present invention also relates to recombinant vectors and recombinant hosts, both prokaryotic and eukaryotic, which contain the substantially purified nucleic acid molecules disclosed throughout this specification. These vectors may be comprised of DNA or RNA; for most cloning purposes DNA vectors are preferred. Typical vectors include plasmids, modified viruses, bacteriophage, cosmids, yeast artificial chromosomes, and other forms of episomal or integrated DNA that can encode a rhBRS-3 protein. It is well within the purview of the skilled artisan to determine an appropriate vector for a particular gene transfer or other use.

An expression vector containing DNA encoding a rhBRS-3-like protein may be used for expression of rhBRS-3 in a recombinant host cell. Such recombinant host cells can be cultured under suitable conditions to produce rhBRS-3 or a biologically equivalent form. Expression vectors may include, but are not limited to, cloning vectors, modified cloning vectors, specifically designed plasmids or viruses. Commercially available mammalian expression vectors which may be suitable for recombinant rhBRS-3 expression, include but are not limited to, pcDNA3. neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565). Also, a variety of bacterial expression vectors may be used to express recombinant rhBRS-3 in bacterial cells. Commercially available bacterial expression vectors which may be suitable for recombinant rhBRS-3 expression include, but are not limited to pCR2.1 (Invitrogen), pET11a (Novagen), lambda gt11 (Invitrogen), and pKK223-3 (Pharmacia). In addition, a variety of fungal cell expression vectors may be used to express recombinant rhBRS-3 in fungal cells. Commercially available fungal cell expression vectors which may be suitable for recombinant rhBRS-3 expression include but are not limited to pYES2 (Invitrogen) and Pichia expression vector (Invitrogen). Also, a variety of insect cell expression vectors may be used to express recombinant protein in insect cells. Commercially available insect cell expression vectors which may be suitable for recombinant expression of rhBRS-3 include but are not limited to pBlueBacIII and pBlueBacHis2 (Invitrogen), and pAcG2T (Pharmingen).

The present invention, therefore, further relates to a process for expressing a rhesus monkey BRS-3 protein in a recombinant host cell, comprising: (a) introducing a vector comprising an isolated nucleic acid molecule into a suitable host cell, the nucleic acid molecule comprising a sequence of nucleotides that encodes a rhesus monkey BRS-3 protein as set forth in SEQ ID NO:2; and, (b) culturing the host cell under conditions which allow expression of said rhesus monkey BRS-3 protein.

Expression of rhBRS-3 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

Recombinant host cells may be prokaryotic or eukaryotic, including but not limited to, bacteria such as E. coli, fungal cells such as yeast, mammalian cells including, but not limited to, cell lines of bovine, porcine, monkey and rodent origin; and insect cells including but not limited to Drosophila and silkworm derived cell lines. For instance, one insect expression system utilizes Spodoptera frugiperda (Sf21) insect cells (Invitrogen) in tandem with a baculovirus expression vector (pAcG2T, Pharmingen). Also, mammalian species which may be suitable and which are commercially available, include but are not limited to, L cells L-M(TK⁻) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

Following expression of rhBRS-3 in a host cell, rhBRS-3 protein may be recovered to provide rhBRS-3 protein in active form. Several rhBRS-3 protein purification procedures are available and suitable for use. Recombinant rhBRS-3 protein may be purified from cell lysates and extracts by various combinations of, or individual application of salt fractionation, ion exchange chromatography, size exclusion chromatography, hydroxylapatite adsorption chromatography and hydrophobic interaction chromatography. In addition, recombinant rhBRS-3 protein can be separated from other cellular proteins by use of an immunoaffinity column made with monoclonal or polyclonal antibodies specific for full-length rhBRS-3 protein, or polypeptide fragments of rhBRS-3 protein.

Another preferred aspect of the present invention is a substantially purified membrane preparation, partially purified membrane preparation, or cell lysate which has been obtained from a recombinant host cell transformed or transfected with a DNA expression vector which comprises and appropriately expresses a complete open reading frame as set forth in SEQ ID NO:1, resulting in a functional form of rhBRS-3. The subcellular membrane fractions and/or membrane-containing cell lysates from the recombinant host cells (both prokaryotic and eukaryotic as well as both stably and transiently transformed cells) contain the functional and processed proteins encoded by the nucleic acids of the present invention. This recombinant-based membrane preparation may comprise a rhesus monkey BRS-3 protein and is essentially free from contaminating proteins, including but not limited to other rhesus monkey source proteins.

Therefore, a preferred aspect of the invention is a membrane preparation which contains a rhesus monkey BRS-3 comprising the functional form of the full length BRS-3 protein as disclosed in FIG. 2 (SEQ ID NO:2). These subcellular membrane fractions will comprise either wild type and/or mutant variations that are biologically functional forms of rhesus monkey BRS-3 at levels substantially above endogenous levels. Any such protein will be useful in various assays described throughout this specification to select for modulators of the rhBRS-3 protein. A preferred eukaryotic host cell of choice to express the rhBRS-3 molecules of the present invention is a mammalian cell line, or Xenopus oocytes.

The DNA molecules, RNA molecules, recombinant protein and antibodies of the present invention may be used to screen and measure levels of rhBRS-3. The recombinant proteins, DNA molecules, RNA molecules and antibodies lend themselves to the formulation of kits suitable for the detection and typing of rhBRS-3. Such a kit would comprise a compartmentalized carrier suitable to hold in close confinement at least one container. The carrier would further comprise reagents such as recombinant rhBRS-3 or anti-rhBRS-3 antibodies suitable for detecting rhBRS-3. The carrier may also contain a means for detection such as labeled antigen or enzyme substrates or the like.

The assays described above can be carried out with cells that have been transiently or stably transfected with rhBRS-3. The expression vector may be introduced into host cells via any one of a number of techniques including but not limited to transformation, transfection, protoplast fusion, and electroporation. Transfection is meant to include any method known in the art for introducing rhBRS-3 into the test cells. For example, transfection includes calcium phosphate or calcium chloride mediated transfection, lipofection, infection with a retroviral construct containing rhBRS-3, and electroporation. The expression vector-containing cells are individually analyzed to determine whether they produce rhBRS-3 protein. Identification of rhBRS-3 expressing cells may be done by several means, including but not limited to immunological reactivity with anti-rhBRS-3 antibodies, and labeled ligand binding.

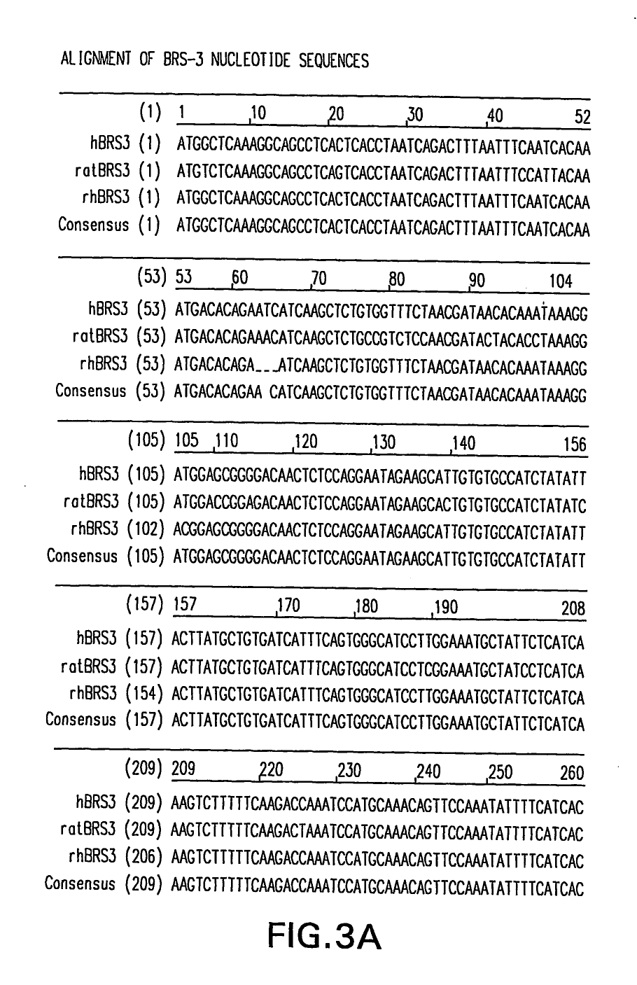

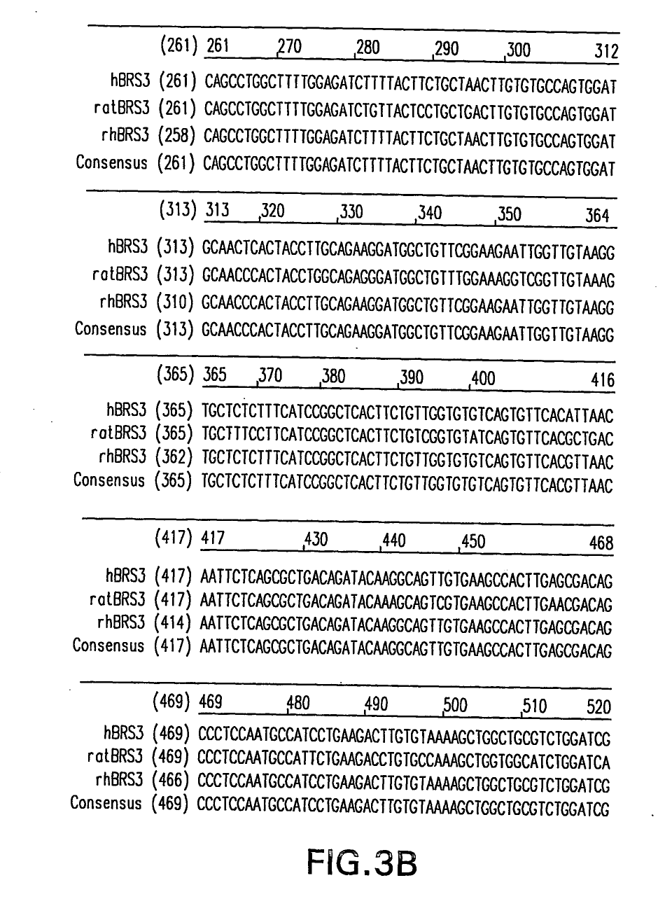

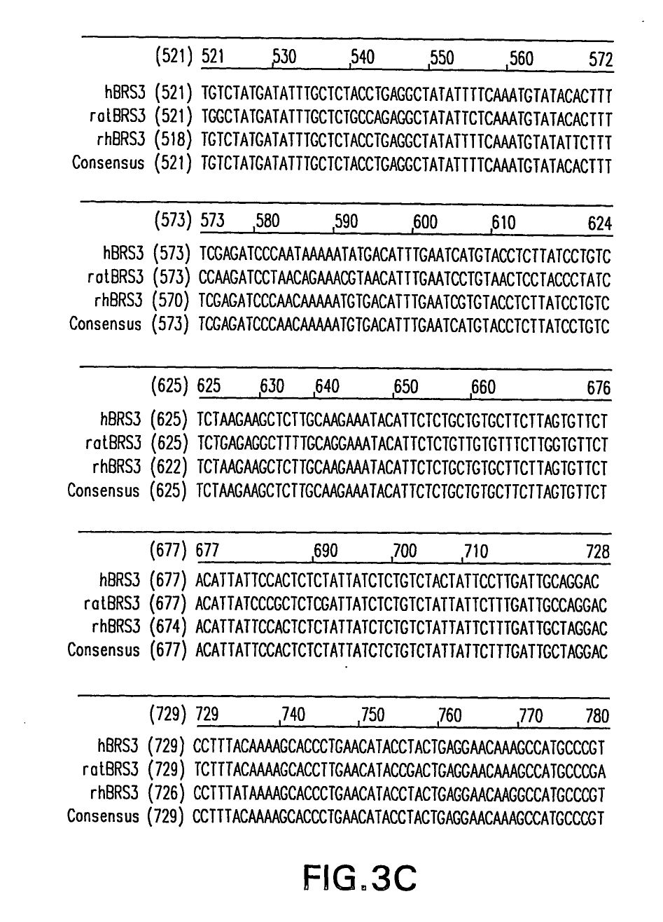

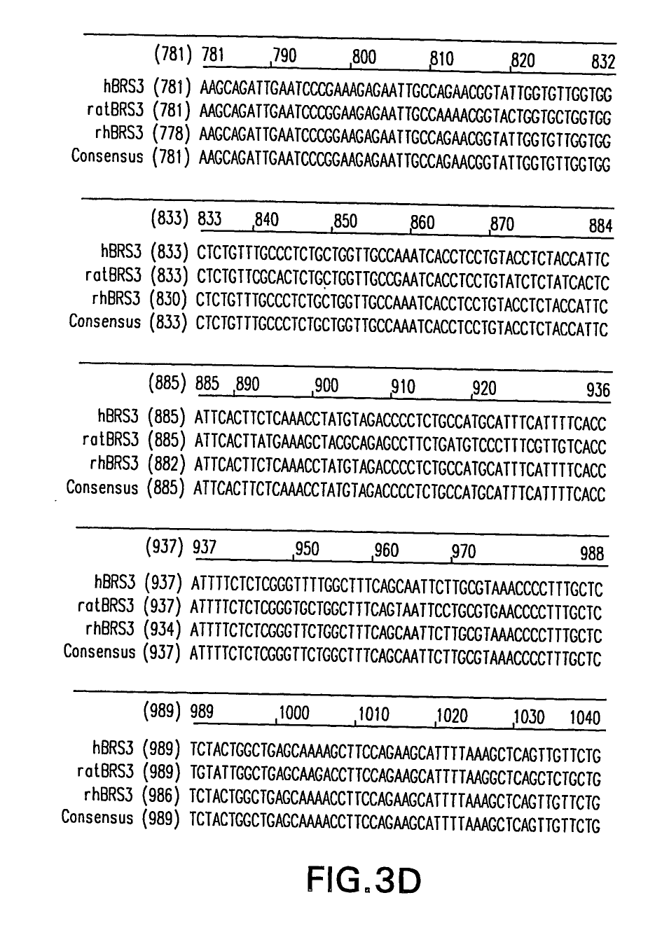

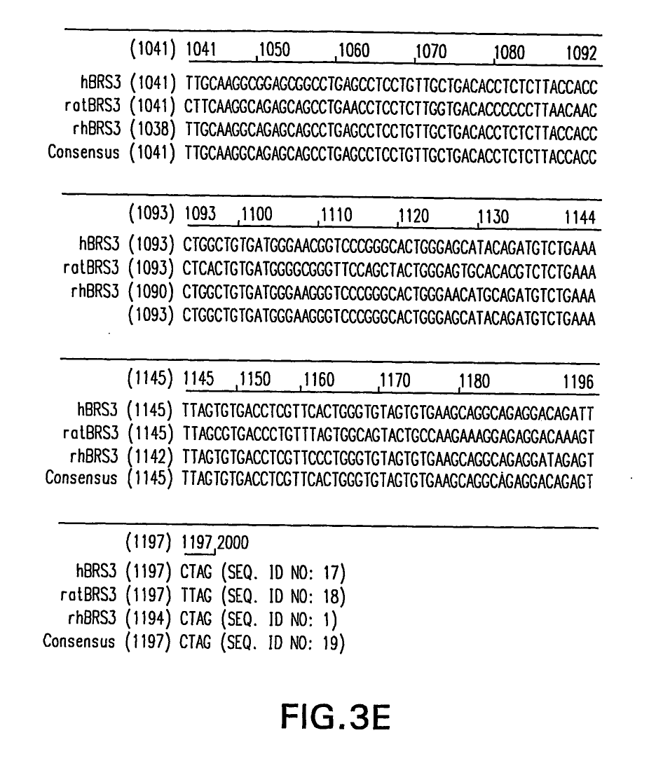

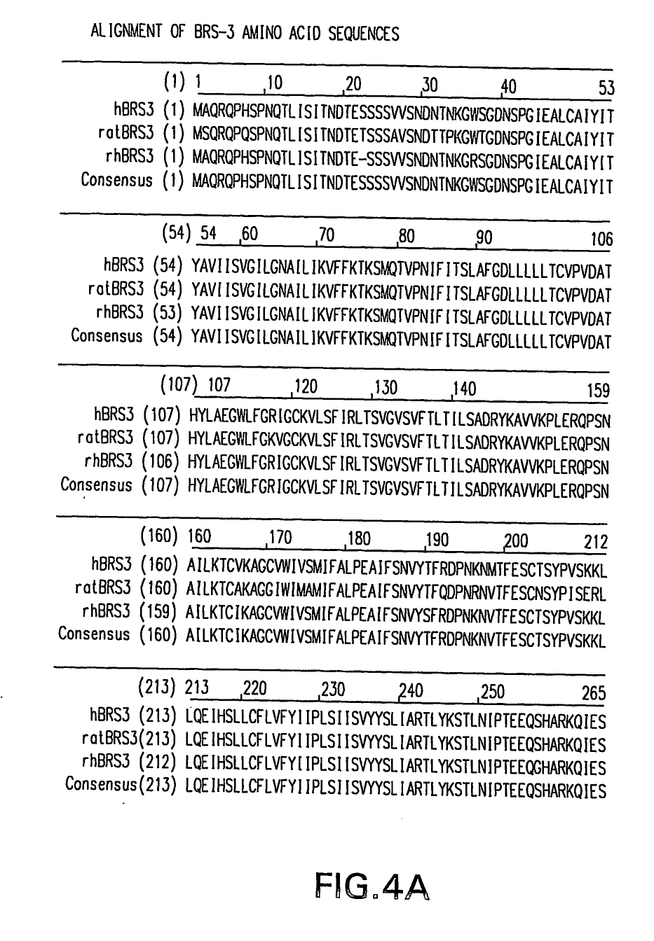

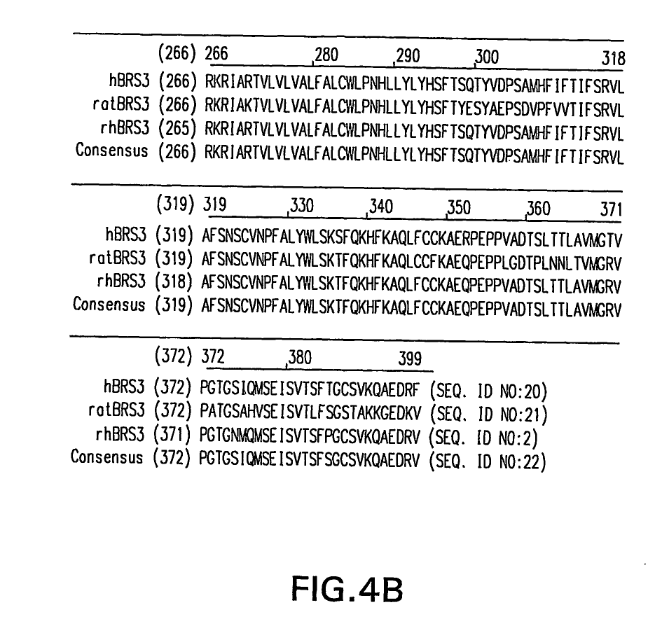

Human BRS-3 has been implicated in the regulation of neuroendocrine function and energy metabolism (Ohki et al. Nature 390: 165-69 (1997)). In addition, mice lacking functional BRS-3 are hyperphagic and have a reduced metabolic rate, which leads to the development of obesity, hypertension and diabetes as adults. The present invention demonstrates that rhesus monkey and human BRS-3 have the same tissue-specific expression patterns (see EXAMPLE 3), and share high sequence similarity (see FIGS. 3 and 4), suggesting an involvement of rhesus monkey BRS3 in energy homeostasis. These observations support the notion that rhesus monkey provides a good animal model to develop BRS-3 agonists as therapeutic agents for obesity.

Accordingly, the present invention is directed to methods for screening for compounds which modulate the expression of DNA or RNA encoding a rhBRS-3 protein as well as compounds which effect the function of the rhBRS-3 protein. Compounds that modulate these activities may be DNA, RNA, peptides, proteins, or non-proteinaceous organic molecules. Compounds may modulate by increasing or attenuating the expression of DNA or RNA encoding rhBRS-3, or the function of the rhBRS-3 protein. Compounds that modulate the expression of DNA or RNA encoding rhBRS-3 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample.

One aspect of this portion of the present invention is a method for identifying compounds that modulate rhesus monkey bombesin receptor subtype-3 expression, comprising contacting a test compound with rhesus monkey bombesin receptor subtype-3, and determining whether the test compound interacts with rhesus monkey bombesin receptor subtype-3.

Methods for identifying agonists and antagonists of other receptors are well known in the art and can be adapted to identify agonists and antagonists of rhBRS-3. For example, Cascieri et al. (1992, Molec. Pharmacol. 41:1096-1099) describe a method for identifying substances that inhibit agonist binding to rat neurokinin receptors and thus are potential agonists or antagonists of neurokinin receptors. The method involves transfecting COS cells with expression vectors containing rat neurokinin receptors, allowing the transfected cells to grow for a time sufficient to allow the neurokinin receptors to be expressed, harvesting the transfected cells and resuspending the cells in assay buffer containing a known radioactively labeled agonist of the neurokinin receptors either in the presence or the absence of the substance, and then measuring the binding of the radioactively labeled known agonist of the neurokinin receptor to the neurokinin receptor. If the amount of binding of the known agonist is less in the presence of the substance than in the absence of the substance, then the substance is a potential agonist or antagonist of the neurokinin receptor. Where binding of the substance such as an agonist or antagonist to rhBRS-3 is measured, such binding can be measured by employing a labeled substance or agonist. The substance or agonist can be labeled in any convenient manner known to the art, e.g., radioactively, fluorescently, enzymatically.

The specificity of binding of compounds having affinity for rhBRS-3 can be shown by measuring the affinity of the compounds for recombinant cells expressing the cloned receptor or for membranes from these cells. Expression of the cloned receptor and screening for compounds that bind to rhBRS-3 or that inhibit the binding of a known, radiolabeled ligand of rhBRS-3 (such as the synthetic peptide, [D-Tyr-betaAla-Phe-Nle] bombesin) to these cells, or membranes prepared from these cells, provides an effective method for the rapid selection of compounds with high affinity for rhBRS-3. Such ligands need not necessarily be radiolabeled but can also be nonisotopic compounds that can be used to displace bound radiolabeled compounds or that can be used as activators in functional assays. Compounds identified by the above method again are likely to be agonists or antagonists of rhBRS-3 and may be peptides, proteins, or non-proteinaceous organic molecules. As noted elsewhere in this specification, compounds may modulate by increasing or attenuating the expression of DNA or RNA encoding rhBRS-3, or by acting as an agonist or antagonist of the rhBRS-3 protein. Again, these compounds that modulate the expression of DNA or RNA encoding rhBRS-3 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample.

Therefore, the present invention provides a method for determining whether a substance is capable of binding to rhesus monkey BRS-3 (rhBRS-3) comprising:

(a) providing test cells by transfecting cells with an expression vector that directs the expression of rhBRS-3 in the cells;

(b) exposing the test cells to the substance;

(c) measuring the amount of binding of the substance to rhBRS-3; and,

(d) comparing the amount of binding of the substance to rhBRS-3 in the test cells with the amount of binding of the substance to control cells that have not been transfected with rhBRS-3.

Also provided herein is a method of identifying a substance which modulates rhBRS-3 receptor activity, comprising: (a) combining a test substance in the presence and absence of a rhBRS-3 receptor protein wherein said rhBRS-3 receptor protein comprises the amino acid sequence as set forth in SEQ ID NO:2; and, (b) measuring and comparing the effect of the test substance in the presence and absence of the rhBRS-3 receptor protein.

This invention further provides a method for determining whether a substance is a potential agonist or antagonist of rhBRS-3 comprising: (a) transfecting or transforming cells with an expression vector that directs expression of rhBRS-3 in the cells, resulting in test cells; (b) allowing the test cells to grow for a time sufficient to allow rhBRS-3 to be expressed; (c) exposing the cells to a labeled ligand of rhBRS-3 in the presence and in the absence of the substance; and, (d) measuring the binding of the labeled ligand to rhBRS-3; where if the amount of binding of the labeled ligand is less in the presence of the substance than in the absence of the substance, then the substance is a potential agonist or antagonist of rhBRS-3.

Pharmaceutically useful compositions comprising modulators of rhBRS-3 may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified rhBRS-3, or either rhBRS-3 agonists or antagonists.

The present invention relates further to transgenic animals, either an invertebrate (e.g., C. elegans) or vertebrate (e.g., mouse), for which the gene encoding rhBRS-3 has been introduced into the germline of the animal. The purpose of this would be to inactivate, in the host, one or several endogenous BRS-3 and observe the biological effects. One such effect may well be an acquired resistance to drugs that are agonists (activators) of BRS-3. In the case of drugs with suspected—but unproven—method of action (MOA) via BRS-3, such BRS-3-harboring transgenic animals may be used to confirm such an effect. Expression of the newly introduced gene encoding rhBRS-3 into the host can be constitutive or inducible, depending on the type of promoter used to drive its expression. Also depending on the type of promoter used, expression of rhBRS-3 can be targeted to a given tissue(s) or it can be generalized.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLE 1

Isolation of Rhesus Monkey BRS-3 cDNA

Poly(A)+mRNA was isolated from rhesus monkey hypothalamus using the FastTrack® 2.0 mRNA isolation kit (Invitrogen, Carlsbad, Calif.) cDNAs were synthesized using SuperScript First-Strand Synthesis System (Invitrogen) with oligo (dT) priming. Using the cDNAs as templates, PCR reactions were carried out with the following primers designed from the 5′ and 3′ untranslated region of the human BRS-3 sequence: BRS3-10 5′-TTGGACGTGACAATCACTGTATTTGAACTGAGA-3′ (SEQ ID NO:3) and BRS3-14 5′-TGTTTCTCCTCCCAGCATGTGTA TCCG -3′ (SEQ ID NO:4). The resulting products were cloned into a pCR4-TOPO vector (Invitrogen). Seven clones were randomly selected from the independent reactions and sequenced using the dye-terminator method with M13 forward and reverse primers as well as with the following olignucleotide primers: P21 5′-GCTCTCTTTCATCCGGCTC -3′ (SEQ ID NO:5), P22 5′-CTGCCTTGTATCTGTCAGCG-3′ (SEQ ID NO:6), P23 5′-CATGCCCGTAAGCAGGTT-3′ (SEQ ID NO:7) P24 5′ -CAGCAGAGGGCAAACA GAG-3′ (SEQ ID NO:8).

One of the above clones, which has a sequence that is identical to the consensus sequence generated from the seven rhesus clones, was chosen as a template for PCR amplification using the following primers:BRS3kozak-F 5′-ATTGGGATCCGCCACCATGGCTCAAAGGCAGCCTCACTCACCT-3′ (SEQ ID NO:9), BRS3Kozak-R 5′-ATGCTCGAGTGGAAAGCTAGACTCTATCCTCTGCCTGC-3′ (SEQ ID NO:10). The amplified products were cloned into the BamHI and EcoRI sites of pcDNA3.1/Hyg (+) (Invitrogen).

### EXAMPLE 2

Poly(A)+mRNA Isolation and cDNA Synthesis

Poly(A)+mRNAs used in this study were isolated from rhesus monkey hypothalamus, cerebellum, pituitary gland, pon, medulla oblongata, liver, brain (except for hypothalamus, cerebellum, pituitary gland, pon, medulla oblongata), liver, and testis using the PastTrack® 2.0 mRNA Isolation Kit (Invitrogen, Carlsbad, Calif.). First strand cDNA was synthesized from 1 ug of mRNA in a 50 μl reaction volume using TaqMan Reverse Transcription Reagents (Applied Biosystems, Foster City, Calif.). Reverse transcriptase reactions were also carried out without template to determine if there was any genomic DNA contamination.

### EXAMPLE 3

Analysis of BRS-3 Expression in Rhesus Monkey by Tag-Man PCR

The distribution of BRS-3 transcripts in adult rhesus monkey was determined using real-time Taq-Man PCR. PCR primers and probes were designed using Primer Express (Applied Biosystems). Primers and probe designed to detect rhesus β-actin were based on the sequence that was originally obtained from rhesus monkey genomic DNA. The following primers and probes were used in the real time PCR: BRS3 primer 1: 5′-(AAAAGAGAGCACCTTACAACCAATT-3′ (SEQ ID NO:11); BRS3 primer2: 5′-CCAGTGGATGCAACCCACTA-3′ (SEQ ID NO:12);FAM-labeled BRS3probe: 5′-TTCCGAACAGCCATCCTTCTGCAAG-3′ (SEQ ID NO:13); β-actin primer 1: 5′-GCAAGCAGGAGTATGACGAGTCT-3′ (SEQ ID NO:14); β-actin primer 2: 5′-AACTAAGTCACAGTCCGCCTAGAAG-3′ (SEQ ID NO:15); VIC-labeled β-actin probe: 5′-CCCTTCCATCGTCCACCGCAAAT-3′ (SEQ ID NO:16). Each of the oligonucleotide fluorescent probes listed above were 3′-labeled with TAMRA (6-carboxytetramethylrhodamine).

Following reverse transcription, the resulting cDNA templates were PCR-amplified in an ABI PRISMS® 7700 Sequence Detection Systems Instrument according to the manufacture's manuscripts (Applied Biosystems, Foster City, Calif.). PCR amplifications were performed in 50 μl reaction volumes containing 0.5 μl of cDNA template, 25 μl of TaqManUniversal PCR Master Mix (Applied Biosystems), 900 nM of each BRS3 and β-actin primer and 250 nM of BRS3 and β-actin probes. The cycling conditions consisted of an initial step of 50° C. for 2 min (UNG incubafion) followed by 95° C. for 10 min (denaturation), and 40 cycles of 95° C. for 15 sec (denaturation) and 60° C. for 1 min (annealing/extension). Expression data were normalized to β-actin expression level.

Taq-man PCR results indicate that BRS3 MnRNA was preferentially expressed in the hypothalamus and testis of rhesus monkeys. Lower levels of expression were detected in other brain regions, including cerebellum, pituitary gland, pons and medulla oblongata. The expression pattern of BRS3 in rhesus monkey mimicked that of the human, suggesting that BRS3 might be involved in energy homeostasis and supporting the notion that rhesus monkey provides a good animal model to develop BRS3 agonists as therapeutic agents for obesity. (Invitrogen, Carlsbad, Calif.). First strand cDNA was synthesized from 1 μg of mRNA in a 50 μl reaction volume using TaqMan Reverse Transcription Reagents (Applied Biosystems, Foster City, Calif.). Reverse transcriptase reactions were also carried out without template to determine if there was any genomic DNA contamination.

### EXAMPLE 3

Analysis of BRS-3 Expression in Rhesus Monkey by Tag-Man PCR

The distribution of BRS-3 transcripts in adult rhesus monkey was determined using real-time Taq-Man PCR. PCR primers and probes were designed using Primer Express (Applied Biosystems). Primers and probe designed to detect rhesus β-actin were based on the sequence that was originally obtained from rhesus monkey genomic DNA. The following primers and probes were used in the real time PCR: BRS3 primer 1: 5′-GAAAGAGAGCACCTTACAACCAATT-3′ (SEQ ID NO:11); BRS3 primer 2: 5′-CCAGTGGATGCAACCCACTA-3′ (SEQ ID NO:12); FAM-labeled BRS3 probe: 5′-TTCCGAACAGCCATCCTTCTGCAAG-3′ (SEQ ID NO:13); β-actin primer 1: 5′-GCAAGCAGGAGTATGACGAGTCT-3′ (SEQ ID NO:14); β-actin primer 2: 5′-AACTAA GTCACAGTCCGCCTAGAAG-3′ (SEQ ID NO:15); VIC-labeled β-actin probe: 5′-CCCTTCCATCGTCCACCGCAAAT-3′ (SEQ ID NO:16). Each of the oligonucleotide fluorescent probes listed above were 3′-labeled with TAMRA (6-carboxytetramethylrhodamine).

Following reverse transcription, the resulting cDNA templates were PCR-amplified in an ABI PRISM® 7700 Sequence Detection Systems Instrument according to the manufacture's manuscripts (Applied Biosystems, Foster City, Calif.). PCR amplifications were performed in 50 μl reaction volumes containing 0.5 μl of cDNA template, 25 μl of TaqManUniversal PCR Master Mix (Applied Biosystems), 900 nM of each BRS3 and β-actin primer and 250 nM of BRS3 and β-actin probes. The cycling conditions consisted of an initial step of 50° C. for 2 min (UNG incubation) followed by 95° C. for 10 min (denaturation), and 40 cycles of 95° C. for 15 sec (denaturation) and 60° C. for 1 min (annealing/extension). Expression data were normalized to β-actin expression level.

Taq-man PCR results indicate that BRS3 mRNA was preferentially expressed in the hypothalamus and testis of rhesus monkeys. Lower levels of expression were detected in other brain regions, including cerebellum, pituitary gland, polls and medulla oblongata. The expression pattern of BRS3 in rhesus monkey mimicked that of the human, suggesting that BRS3 might be involved in energy homeostasis and supporting the notion that rhesus monkey provides a good animal model to develop BRS3 agonists as therapeutic agents for obesity.

### EXAMPLE 4

Functional Expression of Rhesus Monkey BRS3 cDNA.

Measurement of rhesus monkey BRS3 receptor expression in the aequorin-expressing stable reporter cell line 293-AEQ17 (Button et al., 1993. Cell Calcium 14: 663-671) was performed using a Luminoskan RT luminometer (Labsystems Inc., Gaithersburg, Md.) controlled by custom software written for a Macintosh PowerPC 6100. 293-AEQ17 cells (8×10⁵ cells plated 18 hours before transfection in a T75 flask) were transfected with 22 μg of rhesus monkey BRS3 receptor plasmid DNA: 264 μg lipofectamine.

Following approximately 40 hours of expression, the apo-aequorin in the cells was charged for 4 hours with coelenterazine (10 μM) under reducing conditions (300 μM reduced glutathione) in ECB buffer (140 mM NaCl, 20 mM KCl, 20 mM HEPES-NaOH [pH=7.4], 5 MM glucose, 1 mM MgCl₂, 1 mM CaCl₂, 0.1 mg/ml bovine serum albumin). The cells were harvested, washed once in ECB medium and resuspended to 500,000 cells/ml. 100 μl of cell suspension (corresponding to 5×10⁴ cells) was then injected into the test plate containing the BRS3 agonist peptide D-Tyr6-betaAla11-Phe13-Nle14]bombesin6-14, and the integrated light emission was recorded over 30 seconds, in 0.5-second units. 20 μL of lysis buffer (0.1% final Triton X-100 concentration) was then injected and the integrated light emission recorded over 10 seconds, in 0.5-second units.

The “fractional response” values for each well were calculated by taking the ratio of the integrated response to the initial challenge to the total integrated luminescence including the Triton X-100 lysis response. The EC₅₀ value for activation of the rhesus monkey BRS3 receptor was 10 nM. Consistent with results obtained using human BRS-3 (Pradhan et al. Eur J Pharmacol 343: 275-87 (1998); Ryan et al. J Biol Chem 273: 13613-24 (1998)), the synthetic peptide dYB has a nanomolar high affinity to rhesus monkey BRS-3, demonstrating that rhesus monkey BRS-3 is a functional ortholog.

1. An isolated nucleic acid molecule, comprising a sequence of nucleotides that encodes a rhesus monkey BRS-3 protein as set forth in SEQ ID NO:2.

2. The isolated nucleic acid molecule of claim 1 wherein the nucleic acid is DNA.

3. The isolated nucleic acid molecule of claim 1 wherein the nucleic acid is mRNA.

4. The isolated nucleic acid molecule of claim 1 wherein the nucleic acid is cDNA.

5. The isolated nucleic acid molecule of claim 1 wherein the sequence of nucleotides comprises a sequence of nucleotides as set forth in SEQ ID NO:1.

6. An expresion vector comprising the nucleic acid molecule of claim 1.

7. A host cell comprising the vector of claim 6.

8. A subcellular membrane fraction obtained from the host cell of claim 7 which contains recombinant rhesus monkey BRS-3 protein.

9. A process for expressing a rhesus monkey BRS-3 protein in a recombinant host cell, comprising:
(a) introducing a vector comprising the nucleic acid of claim 1 into a suitable host cell; and,
(b) culturing the host cell under conditions which allow expression of said rhesus monkey BRS-3 protein.

10. An isolated and purified rhesus monkey BRS-3 polypeptide comprising a sequence of amino acids as set forth in SEQ ID NO:2.

11. A method for identifying compounds that modulate rhesus monkey bombesin receptor subtype-3 (BRS-3) expression, comprising contacting a test compound with the BRS-3 protein of claim 10, and determining whether the test compound interacts with rhesus monkey bombesin receptor subtype-3.

12. A method for determining whether a substance is capable of binding to rhesus monkey BRS-3 (rhBRS-3) comprising:
(a) providing test cells by transfecting cells with the expression vector of claim 6;
(b) exposing the test cells to the substance;
(c) measuring the amount of binding of the substance to rhBRS-3; and,
(d) comparing the amount of binding of the substance to rhBRS-3 in the test cells with the amount of binding of the substance to control cells that have not been transfected with rhBRS-3.

13. A method of identifying a substance which modulates rhBRS-3 receptor activity, comprising:
(a) combining a test substance in the presence and absence of the rhesus monkey BRS-3 protein of claim 10; and,
(b) measuring and comparing the effect of the test substance in the presence and absence of the rhBRS-3 receptor protein.

14. A method for determining whether a substance is a potential agonist or antagonist of rhBRS-3 comprising:
(a) transfecting or transforming cells with the expression vector of claim 6, resulting in test cells;
(b) allowing the test cells to grow for a time sufficient to allow rhBRS-3 to be expressed;
(c) exposing the cells to a labeled ligand of rhBRS-3 in the presence and in the absence of the substance; and,
(d) measuring the binding of the labeled ligand to rhBRS-3; where if the amount of binding of the labeled ligand is less in the presence of the substance than in the absence of the substance, then the substance is a potential agonist or antagonist of rhBRS-3.
